(19) United States
(12) Patent Application Publication
(10) Pub. No.: US 2021/0290741 A1
(43) Pub. Date: Sep. 23, 2021

(54) COMPOSITIONS FOR THE TREATMENT OF GRAFT VERSUS HOST DISEASE

(71) Applicant: DA VOLTERRA, Paris (FR)

(72) Inventor: Jean DE GUNZBURG, London (GB)

(21) Appl. No.: 17/263,828

(22) PCT Filed: Aug. 5, 2019
(86) PCT No.: PCT/EP2019/071026

§ 371 Date: Jan. 27, 2021

(30) Foreign Application Priority Data
Aug. 5, 2018 (EP) 18187409.0

(51) Int. Cl. A61K 38/50; A61K 33/44; A61K 38/46; A61K 47/64; A61P 37/06

(52) U.S. Cl. CPC A61K 38/50; A61K 33/44; A61P 37/06; A61K 47/64; A61K 38/465

(57) ABSTRACT

The present invention relates to compositions and methods for treatment or prevention of Graft-Versus-Host Disease.

### FIELD OF THE INVENTION

The present invention relates to compositions and methods for treatment and/or prevention of Graft-Versus-Host Disease (GVHD). In particular, the present invention can be used to prevent the antibiotic-triggered disruption of the intestinal microbiota in patients receiving or that will receive a potentially immuno-competent transplantation, such as an allogeneic hematopoietic stem cells transplant, and reduce or prevent the occurrence of GVHD, notably in humans.

### BACKGROUND OF THE INVENTION

Graft-Versus-Host Disease (GVHD) is a major cause of transplantation-associated morbidity and mortality. GVHD can occur after an immuno-competent transplantation. In particular, GVHD can occur after allogeneic cell transplants, such as stem cell transplants and/or bone marrow transplants. GVHD can also occur after blood transfusion. In GVHD, the transplanted immune cells can recognize the cells of the host as foreign and attack them. Patients suffering from GVHD may usually have symptomatic manifestations of three organs such as skin (rash/dermatitis), liver (hepatitis/jaundice), and gastrointestinal tract (abdominal pain/diarrhea). Symptoms can be very pronounced and can even result in host death.

Graft-Versus-Host Disease can be classified largely into acute graft-versus-host disease and chronic graft-versus-host disease. GVHD is acute when it starts within 100 days following the allogeneic hematopoietic stem cell transplant and is usually a reaction of donor immune cells against host tissues. Activated donor T cells typically damage host epithelial cells after an inflammatory cascade that begins with the preparative regimen. Statistically, about 35%-50% of hematopoietic stem cell transplant (HSCT) recipients/patients may develop acute GVHD. The exact risk is usually dependent on the stem cell source, age of the patient, conditioning, and GVHD prophylaxis/treatment used. Acute GVHD is typically staged and graded (grade 0-IV) by the number and extent of organ involvement. Patients with grade III/IV acute GVHD tend to have a poor outcome with a high mortality. Chronic GVHD is the most major and common side effect after HSCT, occurring in 20%-70% of patients living past 100 days following blood and marrow progenitor cell transplantation, and a major cause of death following transplantation.

Currently, standard GVHD preventive protocols are non-specific: immunosuppression by methotrexate, tacrolimus, everolimus, sirolimus, mycophenolate mofetil or cyclosporine A for example which are targeting the proliferating process of immune cells. Further, GVHD treatment typically involves administration of corticosteroids. However, as these immunosuppressive agents have a non-specific effect, they are very toxic and the compromised immune system becomes very sensitive to infectious diseases such as bacterial infections.

Before any HSCT, recipients are put under a preparative regimen (conditioning phase) which puts them in a neutropenic state, hence making them highly susceptible to various infections. In case of fever, when a bacterial infection is feared, patients will be administered an antibiotic treatment to quench the infection at the earliest stage.

Whether antibiotics are administered orally or parenterally, there is a noticeable fraction of the administered dose that reaches the colon in an active form, where it comes into contact with the intestinal microbiota, i.e. the numerous commensal bacteria which live in the gastrointestinal tract, principally in the caecum and colon. As a result of this process, the composition of the microbiota is profoundly altered, affecting both the anaerobic bacteria (that carry out a major physiological role in the intestine of normal subjects and animals) and aerobic bacteria.

In HSCT recipients, dysbiosis, or imbalance of the gut microbiota, has been associated with many diseases, including GVHD. A relationship between microbiota and GVHD has long been suspected but is still not well understood. Some recent works demonstrated a direct link between loss of microbiome diversity and an increase of GVHD severity (Holler, et al. 2014. Biology of Blood and Marrow Transplantation 20(5): 640-645.) and the associated mortality (Taur, et al. 2014. Blood 124(7): 1174-1182.). In addition, more direct evidence for the role of the gut microbiota in the development of GVHD comes from a study in patients showing the association between the increased abundance of commensal bacteria, especially of the Blautia genus, and the reduced lethal incidence GVHD in those patients (Jenq, et al. 2015. Biology of Blood and Marrow Transplantation 21(8): 1373-1383.).

It has also recently been shown that the use of antibiotics, especially broad-spectrum antibiotics, leads to increased GVHD severity and intestinal injury (Shono, et al. 2016. Science Translational Medicine 8(339): 339ra71.). In addition, a recent study highlighted the detrimental role of antibiotics in acute GVHD severity and overall survival (Routy, et al. 2017. Oncolmmunology 6(1): e1258506.).

However, the use of antibiotics is necessary both to prevent and cure bacterial infections in transplanted subjects. Therefore there is a high unmet medical need for pharmaceutically effective solutions to allow the use of antibiotics and prevent and/or treat GVHD in this patient population.

### SUMMARY OF THE INVENTION

The present invention is based on the observation that GVHD correlates with major changes in intestinal microbiota that occur after administration of an immuno-competent graft, such as HSCT, and antibiotic treatments suggesting that antibiotic-associated dysbiosis can be one of the causes of GVHD apparition and/or severity. Antibiotics are not the only pharmaceutical agents that may induce dysbiosis. Preventing the secondary effects of such pharmaceutical agents is thus also desirable.

The present invention therefore provides, among other things, compositions and methods for treating or preventing or reducing risk of GVHD in a subject in need thereof based on the use of a substance suitable for inactivating a dysbiosis-inducing pharmaceutical agent.

In some embodiments, the substance is an adsorbent. In other embodiments, wherein the dysbiosis-inducing pharmaceutical agent is an antibiotic, the substance is an antibiotic-inactivating enzyme.

In one embodiment, the present invention thus relates to an adsorbent, for use in a method for the treatment or the prevention of GVHD, or for reducing the risk or severity of GVHD in a subject.

The invention further relates to an antibiotic-degrading enzyme, for use in a method for the treatment or the prevention of GVHD, or for reducing the risk or severity of GVHD in a subject.

The subject (otherwise referred to as “the host”) may be the recipient of allogeneic cells, tissues or organs, such as cord blood, bone marrow, peripheral blood, stem cells (such as hematopoietic stem cells and adult or embryonic stem cells), blood products and solid organs potentially containing immunocompetent cells.

In some embodiments, the subject is a recipient of a potentially immuno-competent transplantation, such as hematopoietic stem cell (HSCs), bone marrow, peripheral blood (PBSC), and cord blood transplantations.

In some embodiments, the potentially immuno-competent transplantation is cord blood transplantation. In some embodiments, the cord blood transplantation is selected from the group consisting of single cord blood transplantation, double cord blood transplantation, multiple cord blood transplantation manipulated cord blood transplantation, and combination thereof. In some embodiments, the manipulated cord blood transplantation comprises ex vivo expanded cord blood transplantation. In some embodiments, the manipulated cord blood transplantation comprises treatment of the cord blood with prostaglandins prior to transplant. In some embodiments, the manipulated cord blood transplantation comprises depleting T-cells from the cord blood prior to transplant.

In some embodiments, the potentially immuno-competent transplantation is bone marrow transplantation. In some embodiments, the immuno-competent transplantation is peripheral blood transplantation. Suitable bone marrow or peripheral blood may be obtained from either children or adults. Bone marrow and peripheral blood may be manipulated prior to transplantation in any suitable way.

In some embodiments, the potentially immuno-competent transplantation is stem cell transplantation. In some embodiments, the stem cell transplantation is allogeneic stem cell. In some other embodiments, the stem cells are from an adult.

In some embodiments, the potentially immuno-competent transplantation is embryonic stem cell transplantation. In some embodiments, the potentially immuno-competent transplantation is organ transplantation. In some embodiments, the potentially immuno-competent transplantation corresponds to the transfusion of a blood product. Transfusion of a blood product may include, without limitation, blood, serum, plasma and platelet transfusion, as well as the infusion of derived products.

In a particular embodiment, the potentially immuno-competent transplantation is not a human embryonic stem cell.

In another particular embodiment, the subject is an immunocompromised subject. In a further particular embodiment, the subject is immunocompromised by effect of an immunosuppressive treatment or because of a disease, such as an immunodeficiency resulting from a bacterial or viral infection, such as acquired immunodeficiency syndrome (AIDS) that may be the results of an infection by the human immunodeficiency virus (HIV).

In one aspect, the substance is administered at a therapeutically effective amount such that at least one symptom or feature of GVHD is suppressed or reduced in intensity, severity, duration, or frequency or is delayed in onset. In some embodiments, the at least one symptom or feature of GVHD is selected from liver damage, skin rash, jaundice, intestinal inflammation, sloughing of the mucosal membrane, diarrhea, abdominal pain, nausea, and vomiting. In some embodiments, the GVHD is acute GVHD. In some embodiments, the GVHD is chronic GVHD. In some embodiments, the subject being treated is an immune-compromised subject that is susceptible of receiving a transplantation.

In a further particular embodiment, the substance is administered in a therapeutically effective amount during a time sufficient to ensure that GVHD or at least one of its symptoms is suppressed or reduced in intensity, severity, duration, or frequency or is delayed in onset. In particular, the substance may be administered periodically, such as every day, once, twice, three times a day or more than three times a day. For example, the substance may be administered to the subject simultaneously with the dysbiosis-inducing pharmaceutical agent for the same time or several days longer. Illustrative course of treatment include, for example, a dysbiosis-inducing pharmaceutical agent treatment during 7 days, wherein the substance is also administered during the 7 days of exposure to the pharmaceutical agent. In another embodiment, the substance may be administered one or more days before and/or after the onset of therapy with the dysbiosis-inducing pharmaceutical agent to ensure that most of the residual pharmaceutical agent is eliminated. For example, the substance may be administered first the day before the first day of pharmaceutical agent administration, and last two days after the last day of pharmaceutical agent administration.

In the context of the present invention, the substance may be an adsorbent that can be used to adsorb, and therefore remove from the intestine, any residual pharmaceutical agent, or metabolite thereof, after oral or parenteral administration of the pharmaceutical agent, which would otherwise cause adverse effects in the host when it reaches the lower intestine and/or colon.

In a particular embodiment, the subject has received, receives, or will receive an antibiotic for the treatment of an infection. In this embodiment, the substance is administered to prevent the adverse effects of the antibiotic on the intestinal microbiota, in particular in the lower part of the intestine, such as in the late ileum, the caecum or the colon.

In another particular embodiment, the subject does not receive an antibiotic treatment. In this context, the adsorbent is administered to prevent the disruption of the commensal microbiota of the gut for other reasons than for the administration of an antibiotic. For example, the adsorbent may be used to treat an infection from a harmful bacteria, such as from Clostridium difficile, by either impacting the germination or growth of the harmful bacteria, by preventing the production of toxins, or by adsorbing toxins released by such harmful bacteria. In particular, the adverse effects in the host may be caused by any other molecule or toxin that could have serious adverse effects on the intestinal microbiota or on the intestinal tissue, such as, but not limited to, bacterial toxins, and molecules released into the gastro-intestinal tube, including those produced by pathogenic microorganisms.

The substance may be formulated for delivery to a desired part of the intestine. To reduce the concentration of antibiotics, or other molecules with local adverse effects on the intestine or intestinal microbiota, it can be preferable to release the substance at the earliest possible time after the absorption of the antibiotic is complete, with rapid release being preferred. The dosage of the substance is ideally selected to be sufficient to significantly reduce the concentration of the unwanted agent (e.g. antibiotic, other drug, or bacterial or fungal toxins) in the intestine, and also such that the substance remains effective when released. Representative dosage forms include capsules, tablets, pellets and other suitable dosage forms which provide a relatively rapid effect on the removal of the unwanted agent in the colon before the agent can disrupt the intestinal microbiota.

### DETAILED DESCRIPTION OF THE INVENTION

As used herein, the term “acute” when used in connection with tissue damage and related diseases, disorders, or conditions, has the meaning understood by any one skilled in the medical art. For example, the term typically refers to a disease, disorder, or condition in which there is sudden or severe onset of symptoms. In some embodiments, acute damage is due to an ischemic or traumatic event. Typically, the term “acute” is used in contrast to the term “chronic.”

As used herein, the term “chronic,” when used in connection with tissue damage or related diseases, disorders, or conditions has the meaning as understood by any one skilled in the medical art. Typically, the term “chronic” refers to diseases, disorders, or conditions that involve persisting and/or recurring symptoms. Chronic diseases, disorders, or conditions typically develop over a long period of time. The term “chronic” is used in contrast to the term “acute.” In some embodiments, a chronic disease, disorder, or condition results from cell degeneration. In some embodiments, a chronic disease, disorder, or condition results from age-related cell degeneration.

As used herein, the terms “prevent” or “prevention”, when used in connection with the occurrence of a disease, disorder, and/or condition, refer to reducing the risk of developing the disease, disorder and/or condition.

In the context of the present invention, a “risk” of a disease, disorder, and/or condition comprises the likelihood that a particular individual will develop a disease, disorder, and/or condition (e.g., GVHD). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., GVHD). In some embodiments a reference sample or group of reference samples are from individuals comparable to a particular individual. In some embodiments, relative risk is 0, 1, 2, 3, 4, 5, 6, 7, 8, 9, 10, or more.

As used herein, the term “immuno-competent transplantation” denotes a tissue transplantation obtained from a donor, said transplantation containing immunologically competent cells of the donor or containing immunologically competent cells of the donor that could differentiate into immune cells. The immunologically competent cells of the transplantation, such as T cells, may attack and destroy the host cells and thereby induce GVHD.

### GVHD

GVHD is well known in the art (see, for example, Ferrara, J. et al, “Graft-versus-host disease” Lancet. 2009; 373(9674): 1550-61; MacMillan, M. et al., “Acute graft-versus-host disease after unrelated donor umbilical cord blood transplantation: analysis of risk factors,” Blood, 2009, 113(11): 2410-2415; Matsumura, T. et al., “Allogeneic cord blood transplantation for adult acute lymphoblastic leukemia: retrospective survey involving 256 patients in Japan,” Leukemia. 2012 Jan. 17 epub; Kobayashi, K. et al., “Clinical outcomes of unrelated donor umbilical cord blood transplantation for 30 adults with hematological malignancies,” Anticancer Res., 2009 (5): 1763-70)). An individual's immune system functions through recognition of certain cell surface proteins, some of which are termed major histocompatibility complex proteins, or MHC proteins. Additional minor histocompatibility proteins exist which can also contribute to immunological recognition events. The individual mammal's immune system can recognize its own MHC proteins, or those of its identical twin, as self and thus does not destroy its own cells or those of its identical twin. Members of the same species may share major and/or minor histocompatibility antigens, and thus an individual may not recognize the cells of another member of its species as non-self, depending on the degree of the differences between the MHC proteins of the two individuals. When an individual's immune system recognizes the cells of other members of the same species as non-self, the first individual's immune system can proceed to destroy the cells of the second individual. In humans, the major histocompatibility proteins are known as “HLA” antigens.

When tissues such as bone marrow, blood cells, or solid organs are transplanted from one individual to another, the recipient may recognize the donor's cells as non-self and the recipient's immune system may destroy the donor's cells. For this reason, in a tissue transplantation, the recipient can be subjected to immunosuppressive drugs and/or irradiation. However, in GVHD, transplanted patients can also be subject to immunologic recognition in the opposite direction, that is, the donor tissue may contain immunologically competent cells which proceed to destroy the recipient's cells. In particular instances, when a patient receives a HSCT transplant from a healthy donor, the patient's immune system is first ablated by radiotherapy or chemotherapy because the cause of transplant may be the presence of harmful, usually malignant, cells. In this case, the transplanted immune system can recognize as non-self the cells of the patient and attack them.

GVHD can develop when any allogeneic cells, for example cord blood, bone marrow, peripheral blood, adult stem cells, embryonic stem cells, blood products, and/or solid organs containing immunocompetent cells are transferred from a donor to a recipient. Thus, when MHC antigenic differences exist between the donor and recipient, the recipient is at risk for the development of GVHD. T-lymphocytes from the donor recognize the differences based on HLA antigens and attack the new body, i.e., the recipient's body, although most patients and donors are matched as closely as possible for HLA markers. In particular, GVHD results when immunocompetent T cells in the donor graft are infused into an immuno-compromised recipient. Many minor markers, however, differ between donors and patients except when the patient and donor are identical twins. Before a transplant, extensive typing of the donor and recipient is performed to make sure that the donor and recipient are very close immunologically. GVHD may also develop when there are antigenic differences between donor and recipient for the minor histocompatibility antigens. Thus, GVHD can also develop between MHC-matched persons. Moreover, surgery patients who receive directed blood transfusion, for example, transfusion of blood from an HLA homozygous child to a heterozygous parent, may also develop GVHD. In some embodiments, GVHD occurs when the blood is transfused into an immuno-compromised patient (e.g. organ transplant patients on high dose immunosuppressives, children with primary immunodeficiencies, or into HIV infected patients with AIDS).

There are two known forms of GVHD—acute and chronic GVHD. Acute GVHD can occur within the first 100 days following a transplant. Without wishing to be held to a particular theory, it is thought that T-cells present in the donor's tissue and/or cells at the time of transplant can attack the patient's skin, liver, stomach, and/or intestines. The earliest signs of acute GVHD can be a skin rash that appears on the hand, feet and face. Other than blistering skin, patients with severe GVHD can also develop large amounts of watery or bloody diarrhea with cramping from donor T-cells attacking the stomach and intestines. Jaundice (yellowing of the skin and eyes) is a usual indication that GVHD disease involves the liver. The severity of acute GVHD disease can be assessed by the number of organs involved and the degree of symptoms.

Cases of acute GVHD can be categorized into different stages depending on clinical severity (see, for example, Irani, J. et al, “Severe acute gastrointestinal graft-vs-host disease: an emerging surgical dilemma in contemporary cancer care,” Arch Surg. 2008; 143(11): 1041-5). Stage 1 comprises a skin rash over less than 25% of the body. Stage 2 comprises a skin rash over more than 25% of the body accompanied by mild liver or stomach and intestinal disorders. Stage 3 comprises redness of the skin, similar to a severe sunburn, and moderate liver, stomach and intestinal problems. Stage 4 comprises blistering, peeling skin, and severe liver, stomach, and intestinal problems. Additionally or alternatively, acute GVHD can also be characterized into five Clinical Grades 0, I, II, III, and IV. Typically, Grade 0 is substantially symptom free with respect to skin, liver, gut or functional impairment. Grade I is considered mild with skin stage of 1 to 2. Grade II is considered moderate and characterized with skin stage of 1 to 3, liver and gut stage of 1 and functional impairment stage of 1. Grade III is considered severe and characterized with skin stage of 2 to 3, liver and gut stage of 2-3, and functional impairment stage of 2. Grade IV is considered life-threatening and characterized with skin stage of 2 to 4, liver and gut stage of 2 to 4, and functional impairment stage of 3. Exemplary detailed staging and grading are further described in the Examples section.

Chronic GVHD can occur after the first 100 days following a transplant. Without wishing to be held to a particular theory, it is thought that chronic GVHD can be caused by T-cells produced by engrafted tissue and/or cells. The same organs and systems can be attacked as in acute GVHD and additionally chronic GVHD can be associated with damage to connective tissue. Patients with chronic GVHD can experience skin problems that may include a dry itching rash, a change in skin color, and tautness or tightening of the skin. Partial hair loss or premature graying may also occur. Similarly to patients with acute GVHD, patients with chronic GVHD may show jaundice as a sign of liver involvement. Chronic GVHD can also attack glands in the body that secrete mucous, saliva or other lubricants. Patients with chronic GVHD can experience dryness or stinging in their eyes due to impairment of the lacrimal gland. Glands that secrete saliva in the mouth can also be affected by chronic GVHD and, less often, those that lubricate the esophagus, making swallowing and eating difficult. Patients with chronic GVHD can experience a burning sensation in their mouths when using toothpaste or eating acidic foods. Chronic GVHD can attack glands that lubricate the stomach lining and intestines, interfering with the body's ability to properly absorb nutrients. Symptoms can include heartburn, stomach pain and/or weight loss. Occasionally patients with chronic GVHD can experience “contractures,” a tightening of tendons that makes extending or contracting their arms and legs difficult. Chronic GVHD can also affect the lungs, causing wheezing, bronchitis, and/or pneumonia.

The incidence of GVHD can increase with increasing degree of mismatch between donor and recipient HLA antigens, increasing donor age, and increasing patient age. However, the disease may be underdiagnosed and underreported.

As described above, GVHD can occur after a recipient receives a transplant of allogeneic cells, tissues or organs (for example cord blood, bone marrow, adult stem cells, embryonic stem cells, blood products, and/or solid organs).

### Adsorbent and Adsorbent Formulations

The term “adsorbent” designates any compound or material that can adsorb a substrate, typically by physico-chemical binding between the adsorbent surface and the substrate(s) to be adsorbed. Adsorbents may be specific or non-specific. Preferred adsorbents for use in the invention are pharmaceutical grade adsorbents, best suited for use in humans or animals for pharmaceutical or veterinary applications.

Examples of adsorbents suitable for use in the present invention include, without limitation, activated charcoal (also referred to as activated carbon); clays, including bentonite, kaolin, montmorrillonite, attapulgite, halloysite, laponite, and the like; silica, including colloidal silica (Ludox® AS-40 for example), mesoporous silica (MCM41), fumed silica, zeolites and the like; talc; cholesteramine and the like; polystyrene sulfonates and the like; mono and polysulfonated resins; as well as other resins such as those used for bacteriologic testing such as BACTEC® resins.

Preferred adsorbents are activated charcoals (such as from Chemviron, Cabot, Norit, Jacobi Carbons, Merck Millipore, Sigma Aldrich, Desotec, or other sources) which are of pharmaceutical grade. In a particular embodiment, the adsorbent is activated charcoal, more particularly an activated charcoal having a specific surface area above 600 m²/g, in particular above 800 m²/g, in particular above 1000 m²/g, in particular above 1200 m²/g, in particular above 1400 m²/g, in particular above 1600 m²/g, even more particularly above 1800 m²/g. The activated charcoal may be of vegetal, mineral or synthetic origin, its surface being optionally modified by a physical or chemical treatment. In a particular embodiment, the activated charcoal is of vegetal origin. In a particular embodiment, the activated charcoal is derived from peat. In a particular embodiment, the activated charcoal is derived from coconut husks. In a particular embodiment, the activated charcoal is derived from different sources mixed together such as peat and coconut husks. In a particular embodiment, the activated charcoal is characterized by a European molasses number (of note the European molasses number is inversely related to the North American molasses number) which is preferably higher than 100, even more particularly greater than 200, even more particularly greater than 300, even more particularly greater than 400, even more particularly greater than 500, even more particularly greater than 600. In a particular embodiment, the activated charcoal has a phenazone number (measured according to the EU Pharmacopeia) greater than 10 g/100 g, even more particularly greater than 20 g/100 g, even more particularly greater than 30 g/100 g, even more particularly greater than 40 g/100 g, even more particularly greater than 50 g/100 g, even more particularly greater than 60 g/100 g. In a particular embodiment, the activated charcoal is characterized by a density between 0.05 and 0.8, even more particularly between 0.1 and 0.6, even more particularly between 0.15 and 0.5, even more particularly between 0.2 and 0.4.

The amount of adsorbent employed in the methods of the invention may vary depending upon the host/material being treated and the overall capacity, adsorption power and selectivity of the adsorbent. Typically, the amount of adsorbent is an amount sufficient to prevent the deleterious impact of a substance, such as an antibiotic, on the intestinal microbiota known as “dysbiosis” or disruption of the gut microbiota. In particular, the amount of adsorbent is an amount sufficient to prevent or delay the onset of acute and/or chronic GVHD or to reduce the severity of acute and/or chronic GVHD.

The adsorbent for use in the present invention may be formulated in a composition, such as a pharmaceutical composition, which may comprise pharmaceutically acceptable excipients, carriers, and/or additives. Such compositions include formulations for oral delivery, rectal delivery, local application, mucosal application, inhalation, and the like. In a particular embodiment, the adsorbent is formulated in a pharmaceutical composition suitable for administration to humans or animals. More preferably, the adsorbent is formulated in an oral formulation suitable to release said adsorbent in the intestine or in contact with intestinal bacteria, particularly in the gastrointestinal tract, more particularly in the lower part of the intestine, i.e. in the late ileum, the caecum and/or the colon.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Examples of formulations suitable for intestinal delivery of an adsorbent have been described in WO2006/122835 and WO2007/132022. In another embodiment, the adsorbent is formulated in a core. Preferably, the amount of adsorbent is between about 60% and about 100%, more preferably between about 70% and about 98%, more preferably between about 75% and about 95%, more preferably between about 80% and about 90% of the total weight of the core. In a preferred embodiment, the absorbent is formulated with a carrageenan, preferably in the form of a pellet, as proposed in WO2011/104275. Such a formulation can form a core. Such core may be covered with a layer of a coating such that the adsorbent is released in the lower part of the intestine, i.e., in the late ileum, caecum and/or colon. Alternatively, multiple cores may be included or embedded in a dosage unit form suitable for releasing its content in the lower part of the intestine, i.e. in the late ileum, caecum and/or colon, such as a capsule whose shell is suitable for releasing its content in the lower part of the intestine. In another embodiment, the pellets can be included in capsules themselves included in a coated capsule. In another embodiment, the pellets can be included or embedded in Multiple Unit Particle Systems.

Carrageenan is a naturally-occurring family of linear sulphated polysaccharides which are extracted from red seaweeds. Carrageenans are high molecular weight polysaccharides made up of repeating galactose and 3, 6-anhydrogalactose (3,6-AG) units, both sulfated and non-sulfated. The units are joined by alternating alpha 1-3 and beta 1-4 glycosidic linkages. Three basic types of carrageenan are available commercially, i.e. kappa, iota, and lambda carrageenans, which differ by the number and position of the ester sulfate groups on the galactose units. The carrageenan for use in the present invention can be selected from kappa, iota and lambda carrageenans, and mixtures thereof. In one aspect of this embodiment, the adsorbent is mixed with kappa-carrageenan. In a particular embodiment, the mixture comprises activated charcoal and kappa-carrageenan. Preferably, the amount of carrageenan is between about 5% and about 25%, more preferably between about 10% and about 20%, of the total weight of the adsorbent and the carrageenan. In a further particular embodiment, the amount of adsorbent (in particular activated charcoal) in the mixture is between about 95% and about 75%, more preferably between about 90% and about 80%, of the total weight of the adsorbent and the carrageenan. According to a specific embodiment of the invention, the amount of carrageenan is about 15% of the total weight of the adsorbent and the carrageenan. For example, the mixture may contain 85% of an adsorbent and 15% of carrageenan.

According to a particular embodiment of the invention, a mixture of activated charcoal and carrageenan, in particular kappa-carrageenan, is provided with the weight ratios indicated above.

The core (or pellet) may be produced by any suitable means known to the skilled artisan. In particular, granulation techniques are adapted to produce said core. For example, the core may be obtained by mixing the adsorbent and the carrageenan in the ratios indicated above, adding a solvent such as water to proceed to wet granulation, followed by extrusion, optionally followed by spheronization or pelletization with rotary knife, or one-pot pelletization. Any remaining water can be removed, for example, by drying the resulting pellets using conventional techniques.

In one embodiment, the core, or pellet has an average particle size in the range from 50 μm to 6000 μm, in particular 100 μm to 5000 μm, in particular 150 μm to 4000 μm, in particular 250 to 3000 μm, in particular 250 to 1000 μm, in particular 300 to 3000 μm (such as 500 to 3000 μm), in particular 300 to 1000 μm, in particular 500 to 1000 μm, in particular 500 to 700 μm.

The core composition can further include conventional excipients such as anti-adherents, binders, fillers, diluents, flavours, coloration agents, lubricants, glidants, preservatives, sorbents and/or sweeteners. The amounts of such excipients can vary, but are typically in the range of 0.1 to 50% by weight of the pellet.

As discussed above, a preferred formulation of the invention comprises a core comprising an adsorbent, possibly supplemented with carrageenan, which core is covered with a layer of a coating such that the adsorbent is released in the lower part of the intestine, i.e., in the late ileum, caecum and/or colon.

In this regard, in a preferred embodiment, the adsorbent is used as a formulation comprising:
a core containing the adsorbent, and
a layer of an external coating formed around the core such that the adsorbent is released from the formulation in the lower part of the intestine.

In a preferred embodiment, the adsorbent is used as a formulation comprising:
a core containing the adsorbent and carrageenan, and
a layer of an external coating formed around the core such that the adsorbent is released from the formulation in the lower part of the intestine.

Examples of suitable coatings include pH-dependent enterosoluble polymers, azopolymers, disulphide polymers, and polysaccharides, in particular amylose, pectin (e.g. pectin crosslinked with divalent cations such as calcium pectinate or zinc pectinate), chondroitin sulphate and guar gum. Representative pH-dependent enterosoluble polymers include cellulose acetate trimellitate (CAT), cellulose acetate phthalate (CAP), acrylic polymers, methacrylic polymers, anionic copolymers based on methylacrylate, methylmethacrylate and methacrylic acid, hydroxypropyl methylcellulose phthalate (HPMCP), hydroxypropylmethylcellulose acetate succinate (HPMCAS), methacrylic acid and ethyl acrylate copolymers, methacrylic acid and methyl methacrylate copolymers in a 1:1 molar ratio, methacrylic acid and methyl methacrylate copolymers in a 1:2 molar ratio, polyvinyl acetate phthalate (PVAP) and shellac resins. Particularly preferred polymers include shellac, anionic copolymers based on methyl acrylate, methyl methacrylate and methacrylic acid, such as poly(methyl acrylate-co-methyl methacrylate-co-methacrylic acid) in a 7:3:1 molar ratio, as well as methacrylic acid and methyl methacrylate copolymers in a 1:2 molar ratio. Ideally, the polymer dissolves at a pH equal to 6.0 and above, preferably 6.5 and above. Suitable coatings may also be obtained by mixing the polymers and copolymers aforementioned. In another embodiment, suitable coatings are time-dependent coatings or based on time-dependent polymers such as mixture of ethylcellulose polymers with alginate sodiums.

In a particular embodiment, the formulation comprises a further intermediate coating located between the core and the external pH-dependent layer. The intermediate coating can be formed from a variety of polymers, including pH-dependent polymers, pH-independent water soluble polymers, pH-independent insoluble polymers, and mixtures thereof. Examples of such pH-dependent polymers include shellac type polymers, anionic copolymers based on methylacrylate, methylmethacrylate and methacrylic acid, methacrylic acid and ethyl acrylate copolymers, hydroxypropyl methylcellulose phthalate (HPMCP), and hydroxypropylmethylcellulose acetate succinate (HPMCAS). Examples of pH-independent water soluble polymers include PVP or high molecular weight cellulose polymers such as hydroxypropylmethylcellulose (HPMC) or hydroxypropylcellulose (HPC). Examples of pH-independent insoluble polymers include ethylcellulose polymers or ethyl acrylate and methyl methacrylate copolymers.

In a particular embodiment, the invention uses a formulation comprising:
a core comprising a mixture of an adsorbent (preferably activated charcoal) with carrageenan (preferably kappa-carrageenan),
an intermediate coating selected in the group consisting of HPMC, ethylcellulose and a mixture of methacrylic acid and ethyl acrylate copolymer such as Eudragit® L30D-55, and ethyl acrylate and methyl methacrylate copolymer such as Eudragit® NE30D (for example in a mixture weight ratio of 1:9 to 9:1, preferably of 2:8 to 3:7), and
an external layer of an anionic copolymer based on methyl acrylate, methyl methacrylate and methacrylic acid, such as poly(methyl acrylate-co-methyl methacrylate-co-methacrylic acid) 7:3:1, e.g. Eudragit® FS30D.

In a specific embodiment, the formulation comprises a core, comprising about 85% activated charcoal and about 15% kappa-carrageenan, and a coating with an anionic copolymer based on methyl acrylate, methyl methacrylate and methacrylic acid (such as poly(methyl acrylate-co-methyl methacrylate-co-methacrylic acid) 7:3:1, e.g. Eudragit® FS30D, Evonik, Darmstadt, Germany) or a mixture of methacrylic acid and ethyl acrylate copolymer (such as Eudragit® L30D55, Evonik, Darmstadt, Germany).

In another embodiment, the adsorbent is formulated in a composition as disclosed in WO2014044794, comprising:

(a) a core comprising activated carbon;
(b) a first layer around the core, the first layer comprising an insoluble semipermeable material; and
(c) a second layer around the first layer which dissolves at a predetermined pH or which dissolves at a predetermined location in the gastrointestinal tract.

In a variant of this embodiment, the core is activated carbon. In another variant, the activated carbon is sanded or deburred. In yet a further variant, the activated carbon is of particle size 0.02 to 5.0 mm, for example of particle size 0.6 to 1.2 mm. In a further variant, the insoluble semipermeable material comprises one or more of ethyl cellulose, glycerylmonostearate, cellulose acetate butyrate, dipolylactic acid, polyvinyl chloride, and a poly(meth)acrylate polymer such as Eudragit RL 100, Eudragit RL PO, Eudragit RL 30D, Eudragit RL 12.5, Eudragit RS 100, Eudragit RS PO, Eudragit RS 30D, Eudragit RS 12.5 and Eudragit NE 30D, Eudragit HE 40D. In another variant, the first layer further comprises a water soluble material, wherein the first layer may further comprise a water soluble material comprising hydroxypropylmethyl cellulose (HPMC). Said water soluble material may be mixed with the insoluble semipermeable material in certain embodiments and/or may comprise 0.1 to 30% by weight of the amount of the insoluble semipermeable material, for example 2 to 25% by weight of the amount of the insoluble semipermeable material. In a further particular variant, the first layer allows gradual diffusion of molecules through the semipermeable membrane towards the core into contact with the activated carbon. In yet another variant, the second layer comprises a material which dissolves at pH 5 to pH 7. In some variants, the second layer is an enteric layer comprising a material which remains substantially intact at pH 1 to 4.9, but which breaks down rapidly at pH 5 to 7. In a variant, the second layer comprises a pH sensitive polymer. Representative second layers include layers selected from Hypromellose-Acetate-Succinate, cellulose acetate trimellitate (CAT), cellulose acetate phthalate (CAP), anionic copolymers based on methylacrylate, methylmethacrylate and methacrylic acid, hydroxypropyl methylcellulose phthalate (HP CP), hydroxypropylmethylcellulose acetate succinate (HPMCAS), methacrylic acid and ethyl acrylate copolymers, methacrylic acid and ethyl acrylate copolymers, methacrylic acid and methyl methacrylate copolymers (1:1 molar ratio), methacrylic acid and methyl methacrylate copolymers (1:2 molar ratio), Polyvinyl acetate phthalate (PVAP) and Shellac resins. In a further particular variant of this embodiment, the activated carbon is the sole active pharmaceutical ingredient. In still another variant, the composition comprises:

(a) a core comprising activated carbon;
(b) a first layer around the core, the first layer comprising an insoluble semipermeable material in the form of ethyl cellulose, and optionally further comprising a water soluble material comprising hydroxypropylmethylcellulose (HPMC); and
(c) a second layer comprising hydroxypropylmethylcellulose acetate succinate (HPMC AS).

In another variant, the adsorbent is activated carbon formulated in a composition comprising:

(a) a core which is activated carbon;
(b) a first layer around the core, the first layer comprising a semipermeable material which is insoluble in water and further comprises a water soluble material comprising hydroxypropylmethyl cellulose in an amount of 2-25% by weight of the amount of the insoluble semipermeable material; and
(c) a second layer around the first layer which dissolves at pH 5 to 7.

### Antibiotics

The term “antibiotic” designates any compound that is active against bacteria. Antibiotics that may be eliminated or inactivated thanks to the invention include but are not limited to:
beta-lactams including:
   penicillins (such as penicillin G, penicillin V, ampicillin, amoxicillin, bacampicillin, carbenicillin, carbenicillin indanyl, ticarcillin, azlocillin, mezlocillin, piperacillin, and the like),
   penicillinase-resistant penicillins (such as methicillin, oxacillin, cloxacillin, dicloxacillin, nafcillin and the like),
   cephalosporins, such as: first generation cephalosporins (such as cefadroxil, cephalexin, cephradine, cephalothin, cephapirin, cefazolin, and the like); second generation cephalosporins (such as cefaclor, cefamandole, cefonicid, cefoxitin, cefotetan, cefuroxime, cefuroxime axetil, cefinetazole, cefprozil, loracarbef, ceforanide, and the like); third generation cephalosporins (such as cefepime, cefoperazone, cefotaxime, ceftizoxime, ceftriaxone, ceftazidime, cefixime, cefpodoxime, ceftibuten, and the like); fourth generation cephalosporins (such as cefclidine, cefepime, cefozopran, cefpirome, cefquionome and the like); fifth and further generation cephalosporins (such as ceftobiprole, ceftaroline, ceftolozane and the like),
   carbapenems (such as imipenem, meropenem, ertapenem, doripenem and the like)
   monobactams (such as aztreonam, and the like),
quinolones (such as nalidixic acid) and fluoroquinolones (such as cinoxacin, ciprofloxacin, moxifloxacin, levofloxacin, ofloxacin, gatifloxacin, gelifloxacin, norfloxacin and the like),
sulfonamides (e.g., sulfanilamide, sulfadiazine, sulfamethoxazole, sulfisoxazole, sulfacetamide, sulfamethoxydiazine and the like),
aminoglycosides (e.g., streptomycin, gentamicin, tobramycin, amikacin, netilmicin, kanamycin, neomycins B, C and E), spectinomycin, puromycin, gentamicin, and the like),
tetracyclines (such as tetracycline, chlortetracycline, oxytetracycline, methacycline, doxycycline, minocycline, tigecycline, eravacycline and the like),
macrolides (such as erythromycin, azithromycin, clarithromycin, fidaxomicin, telithromycin, josamycin, oleandomycin, spiramycin, tylosin, roxithromycin, cethromycin, solithromycin, and the like),
glycopeptides (such as vancomycin, oritavancin, telavancin, teicoplanin, dalbavancin, ramoplanin and the like),
oxazolidinones (such as linezolid, posizolid, tedizolid, radezolid, cycloserine and the like),
phenicols (such a chloramphenicol, tiamphenicol and the like),
lincosamides (such as clindamycin, lincomycin and the like),
Streptogramins (such as pristinalycin, quinupristin/dalfopristin, virginiamycin and the like)
polymyxins (such as polymyxin A, B, C, D, E1 (colistin A), or E2, colistin B or C, and the like),
diaminopyrimidines (such as trimethoprim, often used in conjunction with sulfamethoxazole, pyrazinamide, and the like),
sulfones (such as dapsone, sulfoxone sodium, and the like),
para-aminobenzoic acid,
bacitracin,
isoniazid,
rifampicins (such as rifampicin, rifabutin, rifapentine, rifalasil, rimamixin, and the like),
ethambutol,
ethionamide,
capreomycin,
clofazimine, and
any other antibacterial agent.

The term “antibiotic” also covers combinations of antibiotics.

### Antibiotic-Inactivating Enzymes and Enzyme Formulations

In certain embodiments, the invention implements antibiotic-inactivating enzymes. In the context of the present invention, an “antibiotic-inactivating enzyme” is an enzyme able to hydrolyse or inactivate an antibiotic, thereby rendering said antibiotic biologically inactive. For example, an antibiotic-inactivating enzyme may substantially increase the minimal inhibitory concentration (MIC) of an antibiotic in comparison to the MIC obtained without said enzyme. According to the present invention, an antibiotic inactivation is total if growth of bacteria, sensitive to a certain concentration of a given antibiotic, in the presence of said concentration of the antibiotic after its treatment with the inactivating enzyme, is identical to growth in the absence of the antibiotic. Another definition of total inactivation is when the MIC of an antibiotic for sensitive bacteria is increased by at least 2 orders of magnitude after treatment with the inactivating enzyme.

Antibiotic-inactivating enzymes for use according to the invention can be natural, chemically modified, genetically engineered or synthetic.

Antibiotic-inactivating enzymes also include functional variants of a parent antibiotic-inactivating enzyme, such as functional variants of a beta-lactamase, erythromycin esterases and ketoreductases. In the context of the present invention, a “functional variant” of an enzyme is an enzyme deriving from a parent enzyme, that has the same type of catalytic activity (for example, a beta-lactamase variant is an enzyme that has beta-lactamase activity), but with a different amino acid sequence. Such a functional variant may have at least 70%, 75%, 80%, 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8%, at least 99.9% identity to the parent enzyme. Such a functional variant may also have a specific activity for a given antibiotic, such as for a given beta-lactam antibiotic in case of a beta-lactamase, of a least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 220%, 240%, 260%, 280%, 300%, 350%, 400%, 500%, 600%, 700%, 800% or even at least 1600%, relative to the specific activity of the parent antibiotic-inactivating enzyme.

Representative antibiotic-inactivating enzymes that may be used in the practice of the present invention include, without limitation, an enzyme inactivating a beta-lactam antibiotic (such as beta-lactamases), an enzyme inactivating a fluoroquinolone (such as aminoglycoside N-acetyltransferases), an enzyme inactivating a macrolide (such as erythromycin-esterases or erythromycin-phosphotransferases), an enzyme inactivating a tetracycline (such as NADPH-dependent oxydoreductase-tetracyclines) or an enzyme inactivating a lincosamide (such as nucleotidyltransferase-lincomycines).

A beta-lactamase is an enzyme (EC 3.5.2.6) having beta-lactamase activity, i.e. an enzyme which catalyzes the irreversible hydrolysis of the amide bond of the beta-lactam ring found in compounds such as beta-lactam antibiotics (e. g. penicillins, cephalosporins, carbapenems, penam sulfones) to create an hydrolyzed molecule devoid of its antibacterial activity. This class of enzymes is well known to those skilled in the art (Wang et al., 1999, Curr Opin Chem Biol. 3(5), 614-22; Frère, J. M. 1995, Mol Microbiol. 16(3):385-95).

In a particular embodiment, the beta-lactamase is a serine beta-lactamase or a zinc-dependent beta-lactamase, also referred to as metallo-beta-lactamase. In another embodiment, the beta-lactamase is selected from class A, class B, class C and class D beta-lactamases. In a further particular embodiment, the beta-lactamase is selected from group 1, group 2, group 3 and group 4 beta-lactamases (Bush et al., Antimicrob. Agents Chemother, 39: 1211). In some embodiments, the beta-lactamase is one or more of P1A, P3A or P4A and their derivatives which consist in derivatives of the beta-lactamase from Bacillus lichenoformis 749/C, or P2A which is the metallo beta-lactamase from Bacillus cereus and derivatives thereof. Furthermore, the beta-lactamase may be an extended-spectrum beta-lactamase (ESBL), optionally selected from a TEM, SHV, CTX-M, OXA, PER, VEB, GES, and IBC beta-lactamase. Further, the beta-lactamase may be an inhibitor-resistant β-lactamase, optionally selected from an AmpC-type β-lactamases, a carbapenemase such as, but not limited toi IMP-type carbapenemases (metallo-β-lactamases), VIM (Verona integron-encoded metallo-β-lactamase) carbapenemases, OXA (oxacillinase) group of β-lactamases, KPC (K. pneumonia carbapenemase), CMY (Class C), SME, IMI, NMC and CcrA, and a NDM (New Delhi metallo-β-lactamase, e.g. NDM-1) beta-lactamases.

In some embodiments, the beta-lactamase is a VIM (Verona integron-encoded metallo-beta-lactamase). Illustrative VIM enzymes include, but are not limited to, VIM-1, VIM-2, VIM-3, VIM-4, and VIM-19. Additional VIM enzymes are described in, for example, Queenan of al. (2007) Clin. Microbiol. Rev. 20(3):440-458. In a further particular embodiment, the beta-lactamase is VIM-2 or a variant thereof. Such beta-lactamases are disclosed in PCT/EP2017/053985, PCT/EP2017/053986 and EP17198414. In specific aspects, the present invention relates to the use of any specific embodiment disclosed in PCT/EP2017/053985, PCT/EP2017/053986 and EP17198414, including any specific variant VIM-2 disclosed therein. In a particular embodiment, the antibiotic-inactivating enzyme is VIM-2, such as represented in SEQ ID NO:1. In another particular embodiment, the antibiotic-inactivating enzyme is a VIM-2 functional variant having an amino acid sequence as shown in SEQ ID NO:2 to 46. In a particular embodiment, the VIM-2 functional variant has a sequences comprising or consisting of SEQ ID NO:29; SEQ ID NO:31, SEQ ID NO:34 or SEQ ID NO: 36.

In another embodiment, the beta-lactamase is the beta-lactamase from Bacillus lichenoformis 749/C or a variant thereof, such as P1A, P3A (also referred to as “ribaxamase”) or P4A. P1A has the sequence shown in SEQ ID NO:47.

In some embodiments, the beta-lactamase is the metallo beta-lactamase from Bacillus cereus (also known as P2A), or a functional variant thereof, as described, for example, in WO2007147945. In a particular embodiment, the P2A enzyme has the sequence shown in SEQ ID NO:48. A functional variant of the P2A enzyme may have at least about 60%, at least about 70%, at least about 80%, at least about 90%, at least about 95%, at least about 97%, at least about 98%, at least about 99%, or 100% identity to sequence shown in SEQ ID NO:48.

In some embodiments, the beta-lactamase is P3A or a functional variant thereof, as described, for example, in WO2011148041. In a particular embodiment, the P3A enzyme has the sequence shown in SEQ ID NO:49 (mature form of the enzyme) or SEQ ID NO:50 (form of the enzyme including a 31 amino acid long signal peptide). A functional variant of the P3A enzyme may have at least about 60%, at least about 70%, at least about 80%, at least about 90%, at least about 95%, at least about 97%, at least about 98%, at least about 99%, or 100% identity to sequence shown in SEQ ID NO:49 or SEQ ID NO:50. In a particular embodiment, the beta-lactamase comprises an amino acid sequence having at least 80% sequence identity with SEQ ID NO:49, and is characterized in that it has a hydrophilic amino acid residue other than aspartic acid (D) at a position corresponding to position 276 according to Ambler classification and said hydrophilic amino acid is selected from arginine (R), histidine (H), lysine (K), asparagine (N), glutamine (Q), serine (S) and threonine (T). In a further particular embodiment, the beta-lactamase comprises an amino acid sequence having at least 80% sequence identity with SEQ ID NO:49, and is characterized in that it has an asparagine (N) at a position corresponding to position 276 according to Ambler classification. In yet another embodiment, the beta-lactamase has the amino acid sequence shown in SEQ ID NO:49, wherein the amino acid residue at the position corresponding to position 276 according to Ambler classification is an asparagine (N).

in another embodiment, the beta-lactamase is P4A or a functional variant thereof, as described, for example, in WO 2015/161243. In a particular embodiment, the P4A enzyme has the sequence of SEQ ID NO:79 or SEQ ID NO:80. A functional variant of the P4A enzyme may have at least about 60%, at least about 70%, at least about 80%, at least about 90%, at least about 95%, at least about 97%, at least about 98%, at least about 99%, or 100% identity to sequence shown in SEQ ID NO:79 or SEQ ID NO:80.

In some embodiments, the beta-lactamase is a Klebsiella pneumoniae carbapenemase (KPC). Illustrative KPCs include, but are not limited to, KPC-1/2 (SEQ ID NO:51), KPC-3 (SEQ ID NO:52), KPC-4 (SEQ ID NO:53), KPC-5 (SEQ ID NO:54), KPC-6 (SEQ ID NO:55), KPC-7 (SEQ ID NO:56), KPC-8 (SEQ ID NO:57), KPC-9 (SEQ ID NO:58), KPC-10 (SEQ ID NO:59), KPC-11 (SEQ ID NO:60), KPC-12 (SEQ ID NO:61), KPC-13 (SEQ ID NO:62), KPC-14 (SEQ ID NO:63), KPC-15 (SEQ ID NO:64), and KPC-17 (SEQ ID NO:65). In an embodiment, the beta-lactamase is KPC-1/2. In an embodiment, the beta-lactamase is KPC-3. The functional variants of KPC enzymes may have at least about 60%, at least about 70%, at least about 80%, at least about 90%, at least about 95%, at least about 97%, at least about 98%, at least about 99%, or 100% identity to the sequences shown in SEQ ID NO:51 to SEQ ID NO:65.

In another embodiment, the beta-lactamase is a New Delhi metallo-beta-lactamase (NDM). Illustrative NDMs include, without limitation, NDM-1 (SEQ ID NO:66), NDM-2 (SEQ ID NO:67), NDM-3 (SEQ ID NO:68), NDM-4 (SEQ ID NO:69), NDM-5 (SEQ ID NO:70), NDM-6 (SEQ ID NO:71), NDM-7 (SEQ ID NO:72), NDM-8 (SEQ ID NO:73), NDM-9 (SEQ ID NO:74), NDM-10 (SEQ ID NO:75), NDM-11 (SEQ ID NO:76), NDM-12 (SEQ ID NO:77), and NDM-13 (SEQ ID NO:78). In an embodiment, the beta-lactamase is NDM-1. In an embodiment, the broad spectrum carbapenemase is NDM-4. The functional variants of NDM enzymes may have at least about 60%, at least about 70%, at least about 80%, at least about 90%, at least about 95%, at least about 97%, at least about 98%, at least about 99%, or 100% identity to sequences shown in SEQ ID NO:66 to SEQ ID NO:78.

In some embodiments, the beta-lactamase is an IMP-type carbapenemase. Illustrative IMP-type enzymes include, without limitation, IMP-1, IMP-4, IMP-8, IMP-11, IMP-43 and IMP-44. Additional IMP-type enzymes are described in, for example, Queenan of al. (2007) Clin. Microbiol. Rev. 20(3):440-458.

In some embodiments, the beta-lactamase from the OXA (oxacillinase) group of beta-lactamases. Illustrative OXA beta-lactamases include, without limitation, OXA-23, OXA-24, OXA-27, OXA-40, OXA-48, OXA-49, OXA-50, OXA-51, OXA-58, OXA-64, OXA-71, and OXA-181. Additional OXA type beta-lactamases are described in, for example, Walther-Rasmussen et al., Journal of Antimicrobial Chemotherapy (2006), 57:373-383 and Queenan et al. (2007) Clin. Microbiol. Rev. 20(3):440-458.

In some embodiments, the beta-lactamase is a CMY (class C carbapenemase) enzyme. An illustrative CMY enzyme with carbapenemase activity is CMY-10, as described in, for example, Lee et al., (2006) Research Journal of Microbiology (1): 1-22.

In some embodiments, the beta-lactamase is a SME enzyme (for Seiratia marcescens). Illustrative SME enzymes include, without limitation, SME-1, SME-2 or SME-3, as described in, for example, Queenan et al. (2007) Clin. Microbiol. Rev. 20(3):440-458.

In some embodiments, the beta-lactamase is an IMI enzyme (imipenem hydrolyzing beta-lactamase). Illustrative IMI enzymes include, without limitation, IMI-1 or IMI-2, as described in, for example, Queenan et al. (2007) Clin. Microbiol. Rev. 20(3):440-458.

In some embodiments, the beta-lactamase is a NMC enzyme (not metalloenzyme carbapenemase). An illustrative NMC enzyme is NMC-A, as described in, for example, Queenan et al. (2007) Clin. Microbiol. Rev. 20(3):440-458.

In some embodiments, the beta-lactamase is a GES enzyme (Guiana extended spectrum). Illustrative GES enzymes include, without limitation, GE-2, GES-4, GES-5, GES-6, GES-7, GES-8, GES-9, GES-11, GES-14 and GES-18 as described in, for example, Queenan of al. (2007) Clin. Microbiol. Rev. 20(3):440-458 and Johnson et al., (2014) Crystal Structures of Class A, B, and D β-Lactamases (http://www.carbapenemase.ca/crystal_structures.html).

In some embodiments, the beta-lactamase is the CcrA (CfiA) metallo-beta-lactamase from Bacteroides fragilis.

In some embodiments, the beta-lactamase is the SFC-1 enzyme from Serratia fonticola or SHV-38 enzyme from Klebsiella pneumoniae, as described in, for example, Walther-Rasmussen et al., (2007) Journal of Antimicrobial Chemotherapy, 60:470-482.

In another embodiment, the antibiotic-inactivating enzyme is an erythromycin esterase. Erythromycin-esterase (EC number 3.1.1) refers to a class of enzymes that catalyze the inactivation of erythromycin as well as other macrolide antibiotics. These enzymes hydrolyze the lactone ring of macrolides such as erythromycin and oleandomycin as explained in Barthelemy et al. 1984, J. Antibiot. 37, 1692-1696. Known erythromycin-esterases are of bacterial origins. They are produced for example by Escherichia coli, Halobacterium salinarum, Gramella forsetii, Achromobacter denitrificans or Rhodococcus sp. In a particular embodiment, the erythromycin-esterase is one of the enzymes usually produced by members of the family Enterobacteriaceae highly resistant to erythromycin as described in Arthur et al. 1987, Antimicrob. Agents Chemother. 31(3), 404-409. Two erythromycin-esterases from E. coli have been documented under the reference names EreA and EreB, the use of both of which being envisioned in the present invention. In a particular embodiment of the invention, the erythromycin-esterase is the EreB erythromycin-esterase from E. coli (cf. Arthur et al. 1986, Nucleic Acids Res 14(12), 4987-4999).

In another embodiment, the antibiotic-inactivating enzyme is a ketoreductase. Ketoreductase (KRED) or carbonyl reductase class (EC 1.1.1.184) enzymes are useful for the synthesis of optically active alcohols from the corresponding prochiral ketone substrate. KREDs typically convert a ketone substrate to the corresponding alcohol product, but may also catalyze the reverse reaction, oxidation of an alcohol substrate to the corresponding ketone/aldehyde product.

In another embodiment, the antibiotic-inactivating enzyme is a hybrid protein molecule. Representative hybrid protein molecules are those disclosed in US Patent Application 20170354706. Such hybrid protein molecule may comprise two enzymes bonded together, capable of inactivating at least one antibiotic. In a particular embodiment, theses enzymes are combined into a single monocatenary protein. These two enzymes can be both from the same class, or each from different classes. For example, the two enzymes can be beta-lactamases, or chosen among the categories of beta-lactamases, enzymes inactivating an aminoglycoside, enzymes inactivating a fluoroquinolone, enzymes inactivating a lincosamide, enzymes inactivating a macrolide, or enzymes inactivating a tetracycline. In a particular embodiment, each enzyme in the hybrid protein molecule inactivates different antibiotics. In another embodiment, the hybrid protein molecule comprises two enzymes capable of inactivating antibiotics belonging to the same class. In a particular embodiment, the sequence of at least one of the component enzymes in the hybrid protein has a sequence homology of at least 40%, 45%, 50%, 55%, 60%, 65%, 70%, 75%, 80%, 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8%, or at least 99.9% with SEQ ID NO:81 to SEQ ID NO:87. In further particular embodiment, the sequence of at least one of the component enzymes in the hybrid protein has a sequence consisting of SEQ ID NO:81, SEQ ID NO:82, SEQ ID NO:83, SEQ ID NO:84, SEQ ID NO:85, SEQ ID NO:86 or SEQ ID NO:87. In a further particular embodiment, the hybrid protein molecule has an amino acid sequence comprising or consisting of a sequence selected in the group consisting of SEQ ID NO:88 to 90.

In another embodiment, the enzyme, whether produced biologically or synthetically, may be further enzymatically and/or chemically modified in order to enhance its activity, stability, solubility or any other beneficial characteristics. One example of such modifications is the linking of polyethylene glycol, or PEGylation, to surface amino groups.

In a particular embodiment, the antibiotic-inactivating enzyme is formulated in a formulation suitable to release the enzyme in a desired part of the intestine. In a particular embodiment, the desired part of the intestine is the lower part of the intestine, such as the ileum, the caecum or the colon. In another particular embodiment, the desired part of the intestine is the upper part of the intestine, such as the duodenum or the jejunum. In a particular embodiment, the formulation comprises pellets of enzymes coated with an enteric coating (such as with an enteric coating dissolving at a pH greater or equal to 7.0). In another particular embodiment, the formulation comprises enteric-coated enzyme pellets (such as with an enteric coating dissolving at a pH greater or equal to 5.5 or at a pH greater or equal to 7.0) within enteric-coated capsules (such as with an enteric coating dissolving at a pH greater or equal to 5.5 or at a pH greater or equal to 7.0). In another particular embodiment, the formulation comprises enteric-coated pellets in uncoated capsules. The choice of the formulation may depend on the route of administration of the antibiotic to the subject. For example, in case of parenteral administration of the antibiotic, a formulation releasing the antibiotic-inactivating enzyme at the upper or lower part of the intestine may be considered. In case of oral administration the antibiotic, the enzyme formulation preferably releases the enzyme in the lower part of the intestine, at a location where the inactivation of the antibiotic by the enzyme cannot interfere anymore with the desired absorption of said antibiotic in the small intestine, in order to benefit from the positive effect of the antibiotic.

### Method of Use

The present invention relates to compositions and methods for treating or preventing or delaying GVHD or reducing the severity of GVHD based on the use of a substance suitable for inactivating a dysbiosis-inducing pharmaceutical agent.

In particular, the present invention can be used to prevent the disruption of the microbiota in patients receiving an allogeneic hematopoietic stem cells transplant and prevent or delay the occurrence of or reduce the severity of GVHD.

In certain aspects, the substance according to the invention is for use in a subject who is administered, will be administered or has been administered with an agent that may disturb the gut microbiota of said subject. Thanks to the invention, the deleterious impact of such agents may be prevented. Therefore, the invention relates to a method for mitigating the deleterious effects a pharmaceutical agent may have on the gut microbiota of a subject who is or could be a recipient of an immuno-competent transplant, comprising administering to said subject an effective amount of a substance according to the invention, suitable for inactivating a dysbiosis-inducing pharmaceutical agent.

The dysbiosis-inducing pharmaceutical agent may be a pharmaceutical agent administered to treat a pathological condition in the subject. Indeed, certain pharmaceutical agents may be administered in order to treat a disease, but may have a deleterious effect on the gut microbiota when they reach the lower part of the intestine. The subject is still to receive the pharmaceutical agent for benefiting its desired effects but, on the other hand, solutions to avoid its secondary effects should be provided. Illustrative pharmaceutical agents having this behavior include antibiotics. As provided above, antibiotics may be administered to a subject in order to treat a bacterial infection. However, since antibiotics are, by design, able to affect bacterial growth or survival, they threaten the gut microbiota balance and may induce dysbiosis when they reach the lower part of the intestine. Other illustrative pharmaceutical agents that may induce dysbiosis include, without limitation:
chemotherapy agents, such as taxanes (e.g. docetaxel, paclitaxel), anthracyclines (e.g. doxorubicin), topoisomerase inhibitors (e.g. etoposide), antimetabolites (e.g. methotrexate, cytarabine, 5-fluorouracil, gemcitabine), alkylating agents (e.g. melphalan), kinase inhibitors (e.g. erlotinib),
antifungal agents, such as voroconazole, ambisome, posoconazole,
antiviral agents, such as acyclovir, methisazone; and
anti-inflammatory agents, such as aspirin, ibuprofen.

Accordingly, in another aspect of the invention the substance according to the invention is administered to a subject who has a cancer and who is treated, will be treated or has been treated with a dysbiosis-inducing pharmaceutical substance, such as an antibiotic.

The adsorbent or the antibiotic-inactivating enzyme (if proper, because the dysbiosis-inducing pharmaceutical substance is an antibiotic) may be administered to the subject even long before transplantation. For example, the subject may have been selected as a transplant recipient but the treatment could not begin before several days, weeks, months or years. In this case, should the subject suffer, between these events, from a disease that would need a treatment with a dysbiosis-inducing pharmaceutical agent, such as an antibiotic, it would be advantageous to prevent gut microbiota dysbiosis by administering an adsorbent or antibiotic-inactivating enzyme as provided herein. Likewise, the adsorbent or the antibiotic-inactivating enzyme may be administered to the subject even long after the day of transplantation. In particular, it may unfortunately be that the subject's transplant be rejected by the host. In this case, halting the systematic administration of an adsorbent or of an antibiotic-inactivating enzyme when the subject receives a dysbiosis-inducing pharmaceutical substance, such as an antibiotic, could severely impair the efficacy of a future transplantation.

In a particular embodiment, the adsorbent or the antibiotic-inactivating enzyme is administered to the subject almost simultaneously with a dysbiosis-inducing pharmaceutical agent, for example an antibiotic. By “almost simultaneously”, it is meant that the adsorbent or the antibiotic-inactivating enzyme is administered shortly before, simultaneously, and/or shortly after administration of the dysbiosis-inducing pharmaceutical agent, in particular an antibiotic, preferably shortly before. In a particular embodiment, the adsorbent or the antibiotic-inactivating enzyme is administered less than 30 minutes before or after the dysbiosis-inducing pharmaceutical agent, in particular an antibiotic, has been administered, in particular less than 20 minutes, less than 19, 18, 17, 16, 15, 14, 13, 12, 11, 10, 9, 8, 7, 6, 5, 4, 3, 2 minutes, or less than one minute before or after the dysbiosis-inducing pharmaceutical agent, in particular an antibiotic, has been administered. In a further particular embodiment, the adsorbent or the antibiotic-inactivating enzyme is administered at least once a day, in particular at least twice a day, more particularly three times a day or four times a day. In a further particular embodiment, the adsorbent or the antibiotic-inactivating enzyme is administered during the whole course of the treatment with the dysbiosis-inducing pharmaceutical agent, in particular with an antibiotic. In a variant of this embodiment, the adsorbent or the antibiotic-inactivating enzyme may be administered a longer time than the dysbiosis-inducing pharmaceutical agent, in particular than an antibiotic, in order to ensure that any residual dysbiosis-inducing pharmaceutical agent, in particular any residual antibiotic, is eliminated. For example, the adsorbent or the antibiotic-inactivating enzyme may still be administered at least one day after, such as two days after interruption of the administration of the dysbiosis-inducing pharmaceutical agent, in particular after the administration of an antibiotic.

In a particular embodiment, the invention relates to an adsorbent or an antibiotic-inactivating enzyme for use in combination with an antibiotic, in particular almost simultaneously, to a subject who is in need of a transplant. According to this embodiment, the adsorbent or the antibiotic-inactivating enzyme prevents the adverse effects the antibiotic could have on the intestinal microbiota of the subject, and therefore may treat or prevent GVHD.

In a specific embodiment, the invention can be used appropriately in patients at risk of GVHD such as patients taking antibiotics waiting for a HSCT procedure, to prevent GVHD occurrence or reduce the severity of a GVHD episode should one episode occur despite the initial treatment with the invention.

In particular, the invention can be used in patients in wait of, or during the course of a HSCT procedure when they receive antibiotics, in particular during the neutropenia phase. The invention can also be used in these patients when they receive antibiotics before the neutropenia phase in order to maintain an optimal microbiota equilibrium. The invention can also be used in patients diagnosed with a cancer of the blood or bone-marrow when they receive antibiotics in order to maintain the microbiota in the best possible state for the longest possible time and improve the outcome of a HSCT if this procedure is deemed necessary to cure the patient.

The invention can also be used in patients having received a HSCT procedure when they receive antibiotics in order to prevent the occurrence of the GVHD syndrome or avoid the worsening of acute or chronic GVHD if the patient already suffers from the disease.

In particular embodiments, the invention can be used every time the subject takes antibiotics. The invention may also be used after the subject has received a fecal microbial transplant or a treatment with probiotics to restore his or her microbiota diversity and is at risk of GVHD.

In a particular embodiment, the subject was administered with an immunosuppressive agent, such as methotrexate, tacrolimus, everolimus, sirolimus, mycophenolate mofetil or cyclosporine A. In another particular embodiment, the subject was administered with an anti-inflammatory drug such as with a corticosteroid.

In a further particular embodiment, the subject has fever. In particular, the antibiotic to be eliminated from the intestine of the subject has been prescribed because of said fever.

In a further particular embodiment, the active compound of the invention is for use in a method for preventing the alteration of the microbiota in a subject who has received, receives or will received an allogeneic transplantation.

The invention can further be used in subjects at high risk of GVHD such as subjects who had a previous episode of GVHD in the years prior to a novel antibiotic cure, a novel hospitalization or a novel immune-suppressive cure.

Thus, the invention thus also relates to a kit comprising an adsorbent and a dysbiosis-inducing pharmaceutical agent, such as an antibiotic, or to a kit comprising or an antibiotic-inactivating enzyme and an antibiotic. The kit may be for use in the treatment or prevention of a pathological condition that may be treated or prevented with the dysbiosis-inducing pharmaceutical agent, such as an antibiotic. In a particular embodiment of the kit, the dysbiosis-inducing pharmaceutical agent is an antibiotic. The kit may further comprise instructions to implement the methods of the present invention, aiming at treating or preventing GVHD. The components of the kit may be administered simultaneously, separately or sequentially. As provided above, the adsorbent or the antibiotic-inactivating enzyme may, in particular, be administered before, during, or after the administration of the dysbiosis-inducing pharmaceutical agent, such as an antibiotic, in particular shortly before or shortly after, more particularly shortly before.

Further aspects and advantages of the invention will be disclosed in the following illustrative experimental section.

### EXAMPLES

### Example 1

In order to evaluate the clinical state of a mouse after a HSCT and noteworthily detect cases of GVHD, a scoring system can be used to monitor multiple clinical symptoms. The score takes into account body weight, the presence of diarrhea, dehydration, depilation and the capacity to move. For each criteria, a score is defined between 0, 1 or 2. The total score is obtained by summing the scores for each criteria. Healthy mice have low total scores whereas unhealthy mice have high total scores with high scores in one or multiple criteria.

If there is no body weight loss or a body weight loss lower than 10%, the score is 0 for this criterion. If the body weight loss is between 10 and 20%, the score is 1. If the body weight loss is higher than 20%, the score is 2.

If the mouse presents no diarrhea, the score is 0. If a slight diarrhea is observed, the score is 1. If the diarrhea is important, the score is 2.

Regarding dehydration, the score is 0 if no sign of dehydration is seen, it is 1 if there is a slight dehydration and it is 2 if the dehydration is important.

If there is no depilation, the score is 0. If there is a light depilation, the score is 1. If the depilation is important, the score is 2.

If the mouse exhibits normal exploratory movements, the score is 0. If the movements of the mouse are limited, the score is 1. If the mouse is almost immobile, the score is 2.

Other criteria could be added to the score if needed such as clinical observations of the eyes, ears, back, fur or other clinically relevant outcomes.

### Example 2

To evaluate the effect of antibiotic use on the development of GvHD following HSCT, female BALB/C mice are irradiated with a γ-source (8 Gy) at Day 0 and injected intravenously with a mix of 5×10⁶ splenocytes and 1×10⁷ bone marrow (BM) cells from C57Bl/6 mice at Day+1. Mice are given antibiotics or placebo from Day-7 to Day+20 by sub-cutaneous administration. During the experiment, survival, body weight and clinical scores, as defined in example 1, are recorded daily until D+30. A high clinical score is related to GVHD complications. On Day+30, a higher disease score is observed in mice receiving the antibiotic compared to mice not receiving the antibiotic.

### Example 3

To evaluate the effect of adsorbents administered with antibiotics on the development of GvHD following HSCT, the same protocol as in example 2 is used, and, for each group treated with antibiotic, another group is added receiving additionally an adsorbent by oral gavage twice a day from Day-7 to Day+25. On Day+30, a lower disease score is observed in mice receiving the adsorbent compared to mice receiving antibiotics without the adsorbent.

### Example 4

To evaluate the effect of antibiotic-inactivating enzymes administered together with antibiotics on the development of GvHD following HSCT, the same protocol as in example 2 is used with a beta-lactam antibiotic. An additional group is introduced, in which animals are also administered a beta-lactamase given by oral gavage, twice a day from Day-7 to Day+25. On Day+30, a lower disease score is observed in mice receiving the beta-lactamase compared to mice receiving the antibiotic without the beta-lactamase.

### Example 5

In order to investigate the effect of an adsorbent on the prevention or attenuation of acute Graft vs Host disease, we performed the following experiment.

30 mice (129, female) were lethally irradiated and transplanted with 5 million C57BL/6 T-cell depleted bone marrow cells as well as 1 million C57BL/6 splenic T cells at day 0.

The mice were then split in 3 groups. In group A, they received no further treatment. In group B, an imipenem treatment was initiated at day 10. It consisted of an administration of imipenem (100 mg/kg), 3 times a week by subcutaneous route. The imipenem treatment was stopped at day 21. In group C, the same imipenem treatment as group B was performed. In addition, group C mice received activated charcoal (14.4 mg/g) mixed with their food in a hydrogel every day from day 9 to day 22.

The clinical status of the mice was monitored every day from day 1 to day 21. In this setting of hematopoietic stem cell transplant, it is known that the mice have a high tendency to develop a lethal acute graft vs host disease.

The percentage of survival in each group at day 21 is presented in the table below:

| Group | Survival at day 21 |
|---|---|
| A (bone marrow + T cells transplant) | 70% |
| B (bone marrow + T cells transplant + | 60% |
| imipenem) | |
| C (bone marrow + T cells transplant + | 90% |
| imipenem + activated charcoal) | |

As presented in the invention, it is seen that the adjunction of an antibiotic to the mice regimen provokes a strengthening of the GvHD condition with a higher mortality. Surprisingly, the addition of activated charcoal, an adsorbent able to capture imipenem in the gut microbiota of mice, is able to revert the deleterious effect of the antibiotic and even improve the outcome of mice by even further reducing the mortality due to Graft vs Host Disease.

1. A method for treating, preventing, delaying or reducing the risk or severity of Graft-Versus-Host disease in a subject comprising administering a substance selected from an adsorbent and an antibiotic-inactivating enzyme to a subject, wherein the subject receives, will receive, or has received a dysbiosis-inducing pharmaceutical agent.

2. (canceled)

3. The method of claim 1, wherein the dysbiosis-inducing pharmaceutical agent is an antibiotic.

4. The method of claim 1, wherein said substance is an adsorbent.

5. The method of claim 4, wherein the adsorbent is activated charcoal.

6. The method of claim 4, wherein the adsorbent is in a formulation comprising:
a core containing the adsorbent, and
a layer of an external coating formed around the core such that the adsorbent is released from the formulation in the lower part of the intestine.

7. The method of claim 6, wherein the core further comprises carrageenan, such as kappa-carrageenan.

8. The method of claim 1, wherein said substance is an antibiotic-inactivating enzyme, wherein the subject receives, will receive or has received an antibiotic for the prevention or the treatment of an infection, and wherein said enzyme is suitable for inactivating said antibiotic.

9. The method of claim 8, wherein (i) the enzyme is a beta-lactamase, in particular VIM-2 or ribaxamase, and the antibiotic is a beta-lactam antibiotic; or (ii) the enzyme is an erythromycin esterase and the antibiotic is a macrolide.

10. The method of claim 8, wherein said enzyme is a hybrid protein molecule comprising two antibiotic-inactivating enzymes bonded together, said two enzymes inactivating the same or different antibiotics or antibiotics from the same or different classes.

11. The method of claim 8, formulated in a composition for oral administration suitable for the release of the antibiotic-inactivating enzyme in a desired part of the intestine, such as in the lower part of the intestine.

12. The method of claim 1, wherein the dysbiosis-inducing pharmaceutical agent is an antibiotic selected in the group consisting of:
beta-lactams including:
penicillins (such as penicillin G, penicillin V, ampicillin, amoxicillin, bacampicillin, carbenicillin, carbenicillin indanyl, ticarcillin, azlocillin, mezlocillin, piperacillin, and the like),
penicillinase-resistant penicillins (such as methicillin, oxacillin, cloxacillin, dicloxacillin, nafcillin and the like),
cephalosporins, such as: first generation cephalosporins (such as cefadroxil, cephalexin, cephradine, cephalothin, cephapirin, cefazolin, and the like); second generation cephalosporins (such as cefaclor, cefamandole, cefonicid, cefoxitin, cefotetan, cefuroxime, cefuroxime axetil, cefinetazole, cefprozil, loracarbef, ceforanide, and the like); third generation cephalosporins (such as cefepime, cefoperazone, cefotaxime, ceftizoxime, ceftriaxone, ceftazidime, cefixime, cefpodoxime, ceftibuten, and the like); fourth generation cephalosporins (such as cefclidine, cefepime, cefozopran, cefpirome, cefquionome and the like); fifth and further generation cephalosporins (such as ceftobiprole, ceftaroline, ceftolozane and the like),
carbapenems (such as imipenem, meropenem, ertapenem, doripenem and the like)
monobactams (such as aztreonam, and the like),
quinolones (such as nalidixic acid) and fluoroquinolones (such as cinoxacin, ciprofloxacin, moxifloxacin, levofloxacin, ofloxacin, gatifloxacin, gelifloxacin, norfloxacin and the like),
sulfonamides (e.g., sulfanilamide, sulfadiazine, sulfamethoxazole, sulfisoxazole, sulfacetamide, sulfamethoxydiazine and the like),
aminoglycosides (e.g., streptomycin, gentamicin, tobramycin, amikacin, netilmicin, kanamycin, neomycins B, C and E), spectinomycin, puromycin, gentamicin, and the like),
tetracyclines (such as tetracycline, chlortetracycline, oxytetracycline, methacycline, doxycycline, minocycline, tigecycline, eravacycline and the like),
macrolides (such as erythromycin, azithromycin, clarithromycin, fidaxomicin, telithromycin, josamycin, oleandomycin, spiramycin, tylosin, roxithromycin, cethromycin, solithromycin, and the like),
glycopeptides (such as vancomycin, oritavancin, telavancin, teicoplanin, dalbavancin, ramoplanin and the like),
oxazolidinones (such as linezolid, posizolid, tedizolid, radezolid, cycloserine and the like),
phenicols (such a chloramphenicol, tiamphenicol and the like),
lincosamides (such as clindamycin, lincomycin and the like),
Streptogramins (such as pristinalycin, quinupristin/dalfopristin, virginiamycin and the like)
polymyxins (such as polymyxin A, B, C, D, E1 (colistin A), or E2, colistin B or C, and the like),
diaminopyrimidines (such as trimethoprim, often used in conjunction with sulfamethoxazole, pyrazinamide, and the like),
sulfones (such as dapsone, sulfoxone sodium, and the like),
para-aminobenzoic acid,
bacitracin,
isoniazid,
rifampicins (such as rifampicin, rifabutin, rifapentine, rifalasil, rimamixin, and the like),
ethambutol,
ethionamide,
capreomycin, and
clofazimine.

13. The method of claim 1, wherein the subject is or could be a recipient of allogeneic cells, tissues or organs.

14. The method of claim 1, wherein the subject is or could be a recipient of hematopoietic stem cells.


SEQUENCE LISTING (90 sequences)

SEQ ID NO: 1 - protein, 240 aa, artificial
  VIM-2
VDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 2 - protein, 235 aa, artificial
  VIM-2 variant
MDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 3 - protein, 235 aa, artificial
  VIM-2 variant
MDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 4 - protein, 235 aa, artificial
  VIM-2 variant
VDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 5 - protein, 235 aa, artificial
  VIM-2 variant
VDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 6 - protein, 240 aa, artificial
  VIM-2  Q34R Q22H
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 7 - protein, 240 aa, artificial
  VIM-2  Q34R Q22N
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 8 - protein, 240 aa, artificial
  VIM-2  Q34R E130D
VDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 9 - protein, 240 aa, artificial
  VIM-2  Q34R E130D Q22N
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 10 - protein, 240 aa, artificial
  VIM-2  Q34R E130D Q22H
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 11 - protein, 235 aa, artificial
  VIM-2  Q34R Q22H DCT236
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 12 - protein, 235 aa, artificial
  VIM-2  Q34R Q22N DCT236
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 13 - protein, 235 aa, artificial
  VIM-2  Q34R E130D DCT236
VDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 14 - protein, 235 aa, artificial
  VIM-2  Q34R E130D Q22N DCT236
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 15 - protein, 235 aa, artificial
  VIM-2  Q34R E130D Q22H DCT236
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 16 - protein, 240 aa, artificial
  VIM-2  V1M Q34R Q22H
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 17 - protein, 240 aa, artificial
  VIM-2  V1M Q34R Q22N
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 18 - protein, 240 aa, artificial
  VIM-2  V1M Q34R E130D
MDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 19 - protein, 240 aa, artificial
  VIM-2  V1M Q34R E130D Q22N
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 20 - protein, 240 aa, artificial
  VIM-2  V1M Q34R E130D Q22H
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 21 - protein, 235 aa, artificial
  VIM-2  V1M Q34R Q22H DCT236
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 22 - protein, 235 aa, artificial
  VIM-2  V1M Q34R Q22N DCT236
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 23 - protein, 235 aa, artificial
  VIM-2  V1M Q34R E130D DCT236
MDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 24 - protein, 235 aa, artificial
  VIM-2  V1M Q34R E130D Q22N DCT236
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 25 - protein, 235 aa, artificial
  VIM-2  V1M Q34R E130D Q22H DCT236
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 26 - protein, 240 aa, artificial
  VIM-2 V10A
VDSSGEYPTASEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 27 - protein, 240 aa, artificial
  VIM-2 V10A Q34R Q22H
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 28 - protein, 240 aa, artificial
  VIM-2 V10A Q34R Q22N
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 29 - protein, 240 aa, artificial
  VIM-2 V10A Q34R E130D
VDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 30 - protein, 240 aa, artificial
  VIM-2 V10A Q34R E130D Q22N
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 31 - protein, 240 aa, artificial
  VIM-2 V10A Q34R E130D Q22H
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 32 - protein, 235 aa, artificial
  VIM-2 V10A Q34R Q22H DCT236
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 33 - protein, 235 aa, artificial
  VIM-2 V10A Q34R Q22N DCT236
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 34 - protein, 235 aa, artificial
  VIM-2 V10A Q34R E130D DCT236
VDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 35 - protein, 235 aa, artificial
  VIM-2 V10A Q34R E130D Q22N DCT236
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 36 - protein, 235 aa, artificial
  VIM-2 V10A Q34R E130D Q22H DCT236
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 37 - protein, 240 aa, artificial
  VIM-2 V10A V1M Q34R Q22H
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 38 - protein, 240 aa, artificial
  VIM-2 V10A V1M Q34R Q22N
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 39 - protein, 240 aa, artificial
  VIM-2 V10A V1M Q34R E130D
MDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 40 - protein, 240 aa, artificial
  VIM-2 V10A V1M Q34R E130D Q22N
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 41 - protein, 240 aa, artificial
  VIM-2 V10A V1M Q34R E130D Q22H
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 42 - protein, 235 aa, artificial
  VIM-2 V10A V1M Q34R Q22H DCT236
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 43 - protein, 235 aa, artificial
  VIM-2 V10A V1M Q34R Q22N DCT236
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 44 - protein, 235 aa, artificial
  VIM-2 V10A V1M Q34R E130D DCT236
MDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 45 - protein, 235 aa, artificial
  VIM-2 V10A V1M Q34R E130D Q22N DCT236
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 46 - protein, 235 aa, artificial
  VIM-2 V10A V1M Q34R E130D Q22H DCT236
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 47 - protein, 263 aa, artificial
  P1A
EMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDDKLIAEATKVVMKALNMNGK

SEQ ID NO: 48 - protein, 215 aa, artificial
  P2A
ETGTISISQLNKNVWVHTELGYFNGEAVPSNGLVLNTSKGLVLVDSSWDNKLTKELIEMVEKKFQKRVTDVIITHAHADRIGGITALKERGIKAHSTALTAELAKNSGYEEPLGDLQTITSLKFGNTKVETFYPGKGHTEDNIVVWLPQYQILAGGCLVKSAEAKDLGNVADAYVNEWSTSIENVLKRYGNINSWPGHGEVGDKGLLLHTLDLLK

SEQ ID NO: 49 - protein, 258 aa, artificial
  P3A variants
  MISC_FEATURE (243)..(243): Xaa can be any naturally occuring amino acid
MKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDXKLIAEATKVVMKALN

SEQ ID NO: 50 - protein, 299 aa, artificial
  P3A variants
  misc_feature (280)..(280): Xaa can be any naturally occurring amino acid
  MISC_FEATURE (290)..(290): Xaa can be any naturally occuring amino acid
MIQKRKRTVSFRLVLMCTLLFVSLPITKTSAQASKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDXKLIAEATKVVMKALNMNGK

SEQ ID NO: 51 - protein, 293 aa, artificial
  KPC-1/2
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPSDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 52 - protein, 293 aa, artificial
  KPC-3
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 53 - protein, 292 aa, artificial
  KPC-4
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKHSEAVIAAAARLALEGLGVNGQ

SEQ ID NO: 54 - protein, 293 aa, artificial
  KPC-5
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 55 - protein, 292 aa, artificial
  KPC-6
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKHSEAVIAAAARLALEGLGVNGQ

SEQ ID NO: 56 - protein, 293 aa, artificial
  KPC-7
MSLYRRLVLLSCLSWPLAGFSATALTNLVAEPFAKLEQDFGGSIGVYAIDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRLRAAVPADWAVGDKTGTCGVYGTANDYAVVWPTGRAPIVLAVYTRAPNKDDKYSEAVIAAAARLALEGLGVNGQ

SEQ ID NO: 57 - protein, 292 aa, artificial
  KPC-8
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKYSEAVIAAAARLALEGLGVNGQ

SEQ ID NO: 58 - protein, 293 aa, artificial
  KPC-9
MSKYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGAYGTANDYAVVWPTGRAPIVLAVYTRAPNKDDKYSEAVIAAAARLALEGLGVNGQ

SEQ ID NO: 59 - protein, 293 aa, artificial
  KPC-10
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 60 - protein, 293 aa, artificial
  KPC-11
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVLWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 61 - protein, 293 aa, artificial
  KPC-12
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELEMNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 62 - protein, 293 aa, artificial
  KPC-13
MSLYRRLVLLSCLSWPLAGFSATALTNLVAEPFAKLEQDFGGSIGVYAMDTGSGATVSYRAEERFPLCSSFKGFLAAAVLARSQQQAGLLGTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 63 - protein, 291 aa, artificial
  KPC-14
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYANDYAVVWPTGRAPIVLAVYTRAPNKDDKHSEAVIAAAARLALEGLGVNGQ

SEQ ID NO: 64 - protein, 292 aa, artificial
  KPC-15
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVLELSAAAVQYSDNAAANLLLKELGGPAKLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKYSEAVIAAAARLALEGLGVNGQ

SEQ ID NO: 65 - protein, 293 aa, artificial
  KPC-17
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQLVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 66 - protein, 269 aa, artificial
  NDM-1
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 67 - protein, 269 aa, artificial
  NDM-2
MELPNIMHPVAKLSTALAAALMLSGCMAGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 68 - protein, 269 aa, artificial
  NDM-3
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTNDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 69 - protein, 268 aa, artificial
  NDM-4
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGLVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 70 - protein, 270 aa, artificial
  NDM-5
MELPNIMHPVAKLSTALAAALMLSGCMPGEIRPTIGQQMETGDQRFGDLVFRQLAPNVWQHTSYLDMPGFGAVASNGLIVRDGGRVLLVDTAWTDDQTAQILNWIKQEINLPVALAVVTHAHQDKMGGMDALHAAGIATYANALSNQLAPQEGLVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 71 - protein, 269 aa, artificial
  NDM-6
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASVRAFGAAFPKASMIVMSHSAPDSRAAITHTARMADKLR

SEQ ID NO: 72 - protein, 268 aa, artificial
  NDM-7
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMNALHAAGIATYANALSNQLAPQEGLVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 73 - protein, 269 aa, artificial
  NDM-8
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMGALHAAGIATYANALSNQLAPQEGLVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 74 - protein, 268 aa, artificial
  NDM-9
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQKGMVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 75 - protein, 269 aa, artificial
  NDM-10
MELPNIMHPVAKLSTALAAALMLSGCMPGEISPTIDQQMETGDQRFGDLVFRQLAPNVWQHTSYLDMPSFGAVTSNGLIVRDGGRVLWDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDRTDIAFGGCLIKDSKAKSLGNLGDADTEHYAASARAFGAAFPKASMIVMSHSAPDSRAAITHTARMADKLR

SEQ ID NO: 76 - protein, 268 aa, artificial
  NDM-11
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGLVAAQHSLTFAANGWEPATAPNFGPLKVFYPGPGHTSDNITVGIDGTDIAFGGCLIKDSKAKSLGNLDDADTEHYAASARAFGAAFPKASMIVMSHSAPDSRAAITHTARMADKLR

SEQ ID NO: 77 - protein, 268 aa, artificial; identical to SEQ ID NO: 76
  NDM-12

SEQ ID NO: 78 - protein, 268 aa, artificial
  NDM-13
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTNDQTAQILNWIKQEINLPVALAWTHAHQDKMGGMDALHAAGIATYANALSNQLAPQEGLVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 79 - protein, 260 aa, artificial
  P4A
EMKDDFAKLEEQFDAKLGIFALDTGTNRTVAYRPDERFAFASTIKALTVGVLLQQKSIEDLNQRITTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVGDKTGSGDYGTRNDIAIIWPPKGDPWLAVLSSRDKKDAKYDNKLIAEATKWMKALNMNGK

SEQ ID NO: 80 - protein, 299 aa, artificial
  P4A + peptide signal
MIQKRKRTVSFRLVLMCTLLFVSLPITKTSAQASKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVGDKTGSGDYGTRNDIAIIWPPKGDPVVLAVLSSRDKKDAKYDNKLIAEATKVVMKALNMNGK

SEQ ID NO: 81 - protein, 263 aa, Escherichia coli
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 82 - protein, 263 aa, Escherichia coli
QTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 83 - protein, 257 aa, Staphylococcus aureus
KELNDLEKKYNAHIGVYALDTKSGKEVKFNSDKRFAYASTSKAINSAILLEQVPYNKLNKKVHINKDDIVAYSPILEKYVGKDITLKALIEASMTYSDNTANNKIIKEIGGIKKVKQRLKELGDKVTNPVRYEIELNYYSPKSKKDTSTPAAFGKTLNKLIANGKLSKENKKFLLDLMLNNKSGDTLIKDGVPKDYKVADKSGQAITYASRNDVAFVYPKGQSEPIVLVIFTNKDNKSDKPNDKLISETAKSVMKEF

SEQ ID NO: 84 - protein, 199 aa, Escherichia coli
MSNAKTKLGITKYSIVTNSNDSVTLRLMTEHDLAMLYEWLNRSHIVEWWGGEEARPTLADVQEQYLPSVLAQESVTPYIAMLNGEPIGYAQSYVALGSGDGRWEEETDPGVRGIDQLLANASQLGKGLGTKLVRALVELLFNDPEVTKIQTDPSPSNLRAIRCYEKAGFERQGTVTTPYGPAVYMVQTRQAFERTRSDA

SEQ ID NO: 85 - protein, 419 aa, Escherichia coli
MRFEEWVKDKHIPFKLNHPDDNYDDFKPLRKIIGDTRVVALGENSHFIKEFFLLRHTLLRFFIEDLGFTTFAFEFGFAEGQIINNWIHGQGTDDEIGRFLKHFYYPEELKTTFLWLREYNKAAKEKITFLGIDIPRNGGSYLPNMEIVHDFFRTADKEALHIIDDAFNIAKKIDYFSTSQAALNLHELTDSEKCRLTSQLARVKVRLEAMAPIHIEKYGIDKYETILHYANGMIYLDYNIQAMSGFISGGGMQGDMGAKDKYMADSVLWHLKNPQSEQKVIVVAHNAHIQKTPILYDGFLSCLPMGQRLKNAIGDDYMSLGITSYSGHTAALYPEVDTKYGFRVDNFQLQEPNEGSVEKAISGCGVTNSFVFFRNIPEDLQSIPNMIRFDSIYMKAELEKAFDGIFQIEKSSVSEVVYE

SEQ ID NO: 86 - protein, 388 aa, Bacteroides fragilis
MTMRIDTDKQMNLLSDKNVAIIGGGPVGLTMAKLLQQNGIDVSVYERDNDREARIFGGTLDLHKGSGQEAMKKAGLLQTYYDLALPMGVNIADKKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKEFSDWDERYKELIHTTLSFVGLATRIFPLEKPWKSKRPLPITMIGDAAHLMPPFAGQGVNSGLVDALILSDNLADGKFNSIEEAVKNYEQQMFMYGKEAQEESTQNEIEMFKPDFTFQQLLNV

SEQ ID NO: 87 - protein, 267 aa, Enterococcus faecium
MLKQKELIANVKNLTESDERITACMMYGSFTKGEGDQYSDIEFYIFLKHSITSNFDSSNWLFDVAPYLMLYKNEYGTEVVIFDNLIRGEFHFLSEKDMNIIPSFKDSGYIPDTKAMLIYDETGQLENYLSEISGARPNRLTEENANFLLCNFSNLWLMGINVLKRGEYARSLELLSQLQKNTLQLIRMAEKNADNWLNMSKNLEKEISLENYKKFAKTTARLDKVELFEAYKNSLLLVMDLQSHLIEQYNLKVTHDILERLLNYISE

SEQ ID NO: 88 - protein, 532 aa, artificial
  TEM36-GGGGGG-CTXM16
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHWGGGGGGQTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 89 - protein, 532 aa, artificial
  fusion CTXM16-6G-TEM36
QTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGLGGGGGGHPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 90 - protein, 537 aa, artificial
  fusion TEM36 - G(EAAAK)2 - CTXM16
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHWGEAAAKEAAAKQTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL